(19) United States
(12) Patent Application Publication
(10) Pub. No.: US 2025/0074659 A1
(43) Pub. Date: Mar. 6, 2025

(54) BAG WITH HIGH WATER VAPOR PROTECTION

(71) Applicant: Fiorini Packaging S.p.A., Senigallia (AN) (IT)

(72) Inventors: Dario Coppola, Arcevia (AN) (IT); Andrea Pierpaoli, Senigallia (AN) (IT)

(73) Assignee: Fiorini Packaging S.p.A., Senigallia (AN) (IT)

(21) Appl. No.: 18/788,324

(22) Filed: Jul. 30, 2024

(30) Foreign Application Priority Data
Sep. 6, 2023 (IT) 102023000018285

(51) Int. Cl. B65D 30/08; B65D 65/42; C07D 275/03; C07D 275/06

(52) U.S. Cl. CPC B65D 31/02; B65D 65/42; C07D 275/03; C07D 275/06; B65D 2565/387

(57) ABSTRACT

The present invention refers to a bag suitable for containing cement, or other similar material, made up of at least two paper sheets superimposed on each other, with a weight of 70-90 g/m², of the semi-extensible type, i.e. with a tensile strength in the machine direction MD of 6.4-7.8 kN/m and a tensile strength in the transverse direction TD of 4.7-5.8 kN/m measured according to ISO 1924-3:2019

Each of said outer paper sheet and internal paper sheet is coated on one side with a water vapor barrier layer having a weight of 10-20 g/m² measured according to the ISO 536:2019 standard,

The barrier layer comprises a styrene-based polymer dispersion and at least one pigment composed of clay and calcium carbonate.

The water vapor barrier layer is in turn coated with at least one hydrophobic paint layer of the water-based wax type, such as for example a polymeric wax, including at least one element of the isothiazol-3-one type, such as example 1,2-benzisothiazol-3 (2H)-one, 5-chloro-2-methyl-2H-isothiazol-3-one (CMIT) and/or 2-methyl-2H-isothiazol-3-one, and mixtures thereof.

In this way, both the transmission of humidity/water vapor (WVTR) and the absorption of water (COBB) are significantly reduced.

In fact, thanks to the water vapor barrier layer and the hydrophobic paint layer applied on it, the water is not retained by the paper, but slides away due to the high surface tension present on the treated paper.

The bag achieves these results despite having no intermediate plastic layers.

### FIELD OF THE INVENTION

The present invention refers to a bag for transporting materials. In particular, the present invention refers to a bag for the transport of cement, or other material useful in various food, chemical and construction sectors, without plastic elements (“plastic free”) and with a high barrier to water vapour.

### BACKGROUND

Currently, for the transport of cement and other similar materials in the construction sector, as well as for the transport of other goods such as in the food, chemical and mineral sectors, bags made of paper and plastic are used.

US 2012/251019 A discloses a recyclable bag for dry based powders, grains, particulates or aggregates having a minimum single ply of high-density porous paper adapted to be foldably configure into a sealable bag shape wherein at least one ply of high density porosity paper has an adhered layer of a hydrophobic biodegradable material on an external and/or internal side wherein the adhered layer of hydrophobic biodegradable material includes a series of holes, spacing and/or slots within or around the hydrophobic biodegradable material to allow air to pass there through for bag aeration during the bag filling.

FR 2970952 A1 describes a bag provided with a valve and having a film comprising a first paper layer, provided with an internal face and an external face. A hydrophobic composition is present on a portion of the outer face of the first paper layer such that the coated portion of the outer face has an air wetting tension less than or equal to the water wetting tension. The film comprises a second paper layer, which is fixed to the inner face of the first paper layer by a layer of glue dots forming an adhesive layer.

DE 202014000194U describes a bag that has at least one paper layer having an overlap which is sealingly adhesively bonded toward the sack interior; and wherein the layer of the paper layer facing the sack interior has an air-permeable region in the region of the overlap which is covered by the layer of the paper layer facing the sack exterior. The at least one paper layer at least regionally has a water-repellent/water-tight coating facing the sack exterior.

Typically, the known in the art bags are made up of two overlapping paper sheets of, for example, 80 g/m², between which a plastic sheet, typically a high-density polyethylene (HDPE), or with a density in the range between 0.945 and 0.965 g/cm3 and a thickness, for example, ranging from 7 microns to 12 microns, is interposed.

These bags show reasonable resistance to humidity, even if stored for a long time in a humid environment.

However, these bags have the defect of containing plastic elements; considering the very large number of bags of this type used throughout the world for the various material transportation, the fact of containing plastic elements constitutes a serious problem from an environmental point of view.

The Italian patent application published with n. IT 102020000013471 in the name of the same applicant of the present application describes a bag without plastic elements consisting of at least two paper sheets superimposed on each other, wherein the external sides of said sheets are coated with at least one hydrophobic paint layer. This allows the bag to have better protection against moisture.

Available on the market bags made up of at least one sheet of semi-extensible paper on which a layer of protection against water vapor is applied are also known; the presence of this protective layer allows the bag to have a significantly improved barrier against humidity.

However, the Applicant has found the need to create bags, free of plastic elements, which show even more efficient protection against water vapor compared to prior art bags.

Therefore, the Applicant has faced the technical problem of how to effectively and economically create plastic-free bags for the transport of cement, glue and other similar material, which are still able to preserve from humidity the material contained therein.

### SUMMARY

The present invention refers to a bag as the one indicated in claim 1.

The Applicant of the present application has in fact surprisingly found that the cited above technical problem can be effectively and reliably solved by using a bag made up of at least two paper sheets (an outer paper sheet and an internal paper sheet) superimposed on each other, wherein:
each of said outer paper sheet and internal paper sheet has a weight of 70-90 g/m² and is of the semi-extensible type, i.e. it has a tensile strength in the machine direction MD of 6.4-7.8 kN/m and a tensile strength in the transverse direction TD of 4.7-5.8 kN/m measured according to ISO 1924-3:2019,
each of said outer paper sheet and internal paper sheet is coated on one side with a water vapor barrier layer having a weight of 10-20 g/m² measured according to the ISO 536:2019 standard,
said barrier layer comprises a styrene-based polymer dispersion and at least one pigment composed of at least clay and calcium carbonate,
said water vapor barrier layer is in turn coated with at least one hydrophobic paint layer of the water-based wax type, such as for example a polymeric wax, including at least one element of the isothiazol-3-one type, such as for example 1,2-benzisothiazol-3 (2H)-one, 5-chloro-2-methyl-2H-isothiazol-3-one (CMIT) and/or 2-methyl-2H-isothiazol-3-one, and their mixtures.

In this way, both the transmission of humidity/water vapor (WVTR) and the absorption of water (COBB) are significantly reduced.

In fact, thanks to the water vapor barrier layer and the hydrophobic paint layer applied onto it, the water is not retained by the paper sheets, but slides away due to the high surface tension present on the treated paper.

This results in a highly hydrophobic surface in the broadest sense of the term, to indicate the property of not absorbing or retaining water.

The barrier effect is thus increased compared to that one obtainable from standard known in the art plastic bags, guaranteeing high humidity protection to the product contained inside the bag, even in the absence of intermediate polythene plastic sheets in the bag construction, and consequently improving the shelf life of the product contained inside the bag.

In particular, the bag of the present invention appears to be free of plastic elements (“plastic free”), while maintaining high shelf conservation performance against humidity and water.

Furthermore, in this way, the paper sheets that make up the bag are given an excellent smoothness characteristic of the Bentsen type, measured according to the UNI ISO 8791-2:2013 standard with values ranging from 82 to 88 ml/min.

The term “bag” in this description and in the attached claims refers to a sack, bag or any container or packaging of the roll-on-reel type that can be printed with inks and paints, suitable for safely transporting and keeping intact the material contained at its inside, this material being of solid, semi-solid, or granular form, and of the various product classes such as, for example, earth, sand, cement, stucco, or other material useful in the construction sector, as well as fodder, seeds, food for animals and food for people, or other material useful in the food, chemical or for the transport of minerals and similar sectors, and this container being such that it can assume the shape determined by the material contained in its entirety within it.

According to a preferred embodiment, said at least one hydrophobic paint layer has a basis weight of 4-9 g/m².

According to a preferred embodiment, said hydrophobic paint has a pH between 8 and 9.

According to a preferred embodiment, said hydrophobic paint has a relative density of approximately 1 g/cm³ at 20° C.

According to a preferred embodiment, said hydrophobic paint has a viscosity <2000 mPas, preferably <1500 mPas.

According to a preferred embodiment, said paint is distributed on each of said sheets by means of the spreading process, i.e. an industrial process including the uniform application of said paint on said sheets, drying through an oven, possible calendering or embossing, and subsequent cooling.

According to a preferred embodiment, said outer paper sheet of the semi-extensible type has a tensile index in the machine direction MD equal to approximately 75 Nm/g according to the ISO 1924-3:2019 standard.

According to a preferred embodiment, said outer paper sheet of the semi-extensible type has an elongation at break in the machine direction MD equal to approximately 5.2% and an elongation in the transverse direction TD of 9-9.5% according to the standard ISO 1924-3:2019.

According to a preferred embodiment, each of said outer paper sheet and internal paper sheet is coated on one side with a water vapor barrier layer having a weight of approximately 15 g/m².

In this way, further protection against water vapor is obtained.

According to a preferred embodiment, said barrier layer comprises a polymer dispersion comprising styrene-acrylate and/or styrene-butadiene.

According to a preferred embodiment, said barrier layer is applied to said outer paper sheet and to said internal paper sheet by means of a coating machine in off-line mode.

According to a preferred embodiment, said coating machine is equipped with a blade and an air dryer.

According to a preferred embodiment, said bag is constituted by a main lateral external surface and by at least one lower bottom fixed to said main external surface, wherein each of said main lateral external surface and said lower bottom is constituted by at least two of said paper sheets superimposed on each other coated with at least one hydrophobic paint layer.

In this way the bag appears as if it were made up of a single body.

Preferably, the consistency of the external lateral surface of the bag is such as to allow the bag itself to assume the shape defined by the mass of the material contained therein.

Preferably, said lower bottom has a flat, partially rigid surface, such that it can act as a support base on the ground for the bag. In this way, since this lower bottom has a substantially flat dimension, it is easier to place the bag on the ground on flat surfaces in a vertical position when the bag is full, or partially full.

In one embodiment, preferably, said bag also consists of an upper bottom, substantially similar to the lower bottom; in this way it is possible to place the bag on the ground both using the lower base as a support base on the ground and, vice versa, using the upper base as such a support base. This is particularly useful when the bag to be placed on the ground does not have one part that must necessarily face upwards and one facing upwards.

In an alternative embodiment, the bag of the present invention is of the type defined as “open mouth”, where said upper bottom is not present; the bag in this alternative embodiment is in fact presented with the upper part of the lateral surface open so that it can be easily filled. Once the bag has been filled, this opening is closed with special sealing machines.

According to a preferred embodiment, the internal sides of said at least two sheets are glued to each other at the upper end and the lower end of said sheets so as to create a sort of tube inside the bag.

According to a preferred embodiment, the internal sides of said at least two sheets are glued to each other at points arranged in correspondence with said upper and lower ends. According to a preferred embodiment, the internal sides of said at least two sheets are glued to each other using starch glue or vinyl glue.

Further characteristics and advantages of the present invention will be better highlighted by examining the following detailed description of preferred, but not exclusive, embodiments, illustrated for indicative and non-limiting purposes only.

### DETAILED DESCRIPTION

The following detailed description refers to particular embodiments of the device of the present invention, without limiting its content.

The bag of the present invention is suitable for containing cement, or other similar material useful in the construction industry, or in the food sector, minerals, chemicals, such as sand, gravel, or other useful material, for example, in the feed and seed sector for animals or similar.

### Example 1 (Comparison)

A known in the art bag, here called CFR1, is made up of two paper sheets, defined as outer sheet and internal sheet, superimposed on each other, in the absence of plastic elements; the outer sheet is a semi-extensible paper sheet having a weight of 70 g/m² coated, on one of its two sides, with a water vapor barrier layer having a weight of 15 g/m², measured according to the ISO 536:2019 standard. The water vapor barrier layer comprises a styrene-based polymer dispersion and at least one pigment mainly composed of clay and calcium carbonate. In this case, a total weight of 85 g/m² is therefore obtained. The structure of the outer sheet of the CFR1 comparison sample is therefore as follows:

water vapor barrier layer
outer sheet of semi-extensible paper

Furthermore, the outer sheet has a tensile strength in the machine direction MD of 6.4-7.8 kN/m and a tensile strength in the transverse direction TD of 4.7-5.8 kN/m, and a traction in machine direction MD equal to approximately 75 Nm/g according to ISO 1924-3:2019. The outer sheet also has an elongation at break in the machine direction MD of approximately 5.2% and an elongation in the transverse direction TD of 9-9.5%, also measured according to ISO 1924-3:2019. The outer sheet is overlaid on an internal sheet of standard semi-extensible paper (i.e., without the water vapor barrier layer). The outer sheet has a width that extends at least 20 mm compared to the width of the internal sheet, to facilitate longitudinal gluing.

The CFR1 comparison bag has a main lateral external surface, a lower bottom and an upper bottom fixed to the main external surface, so as to be made up of a single body. The lower bottom and the upper bottom include a flat, partially rigid surface, such that it can be folded onto the lateral surface when the bag is empty, so as to be able to store the bag taking up the minimum necessary space, and such that it can act as the base of the bag, when it is at least partially full and placed in a vertical position.

### Example 2 (Comparison)

Another known in the art bag (see the Italian patent application published with no. IT 102020000013471 in the name of the same applicant of the present application), here called CFR2, is similar to the CFR1 comparison bag seen above, with the difference that in the CFR2 comparison bag there is no water vapor barrier layer applied on the outer sheet, onto which instead a hydrophobic paint layer of the water-based polymer wax type is applied, with a weight of 5 g/m², a pH of approximately 8.5, a relative density of approximately 1 g/cm³ at 20° C. and a viscosity <1,500 mPas. The structure of the outer sheet of the CFR2 comparison sample is therefore as follows:

hydrophobic paint layer
outer sheet of semi-extensible paper

### Example 3 (Invention)

One bag of the invention, herein referred to as INV1, comprises the same semi-extensible paper outer sheet used in the CFR1 and CFR2 comparison examples, coated with the same water vapor barrier layer used in the CFR1 comparison example, with the further addition on the latter of the same hydrophobic paint layer used in the CFR2 comparison example. The structure of the outer sheet of the INV1 sample is therefore as follows:

hydrophobic paint layer
water vapor barrier layer
sheet of special semi-extensible paper

### Test.

The comparison CFR1 and CFR2 bags and the INV1 invention bag were filled with cement, or other similar material as indicated above, and subsequently suitably closed according to the traditional methods of filling and closing these types of bags. For example, the cement can be inserted inside each bag through an opening made in the lower bottom, an opening which is subsequently automatically closed at the end of the bag filling phase, via a particular valve the lower bottom is provided with. The bags thus filled and subsequently closed were subjected to the atmospheric agent action for a period of 7 days, thus simulating the finished product storage condition by an end user. The humidity to which the bags were exposed is the environmental humidity.

Test 1: to determine the paper water absorption, the Cobb method described in the European standard EN ISO 535 was used, using 3 different contact times (60 seconds, 120 seconds, 300 seconds) and expressing the results in g/m².

Test 2: to determine reliable values of the water vapor transmission (WVTR) through the paper sheets, the test method described in ASTM E96//E96M-15 procedure E was used, both in the standard environmental condition of 23° C. and 50% relative humidity, and in borderline, tropical conditions of 38° C. and 90% relative humidity, expressing the results in g/m² for 24 hours.

Table 1 below shows the results of the various tests.

TABLE 1

| | | | | | Test 2 | Test 2 |
|---|---|---|---|---|---|---|
| | | | | | 23° C. | 38° C. |
| | | Test 1 | Test 1 | Test 1 | 50% HR | 90% HR |
| | | 60 sec. | 120 sec | 300 sec | g/m² for | g/m² for |
| Sample | Structure | g/m² | g/m² | g/m² | 24 hours | 24 hours |
| CFR1 | Semi-extensible paper + | 31.1 | 45.8 | 49.6 | 55 | 824-962 |
| (comparison) | barrier layer | | | | | |
| CFR2 | Semi-extensible paper + | 4.7 | 5.5 | 5.6 | 115 | 735-757 |
| (comparison) | paint layer | | | | | |
| INV1 | Semi-extensible paper + | 10.1 | 11.4 | 12.3 | 15 | 255-292 |
| (invention) | barrier layer + paint layer | | | | | |

From Table 1 it can be seen that the CFR2 comparison sample showed a water vapor transmission (WVTR) value through the paper sheets equal to 115 g/m² for 24 hours in the standard environmental condition of 23° C. and 50% of relative humidity (Test 2). By using the CFR1 comparison sample, this water vapor transmission value was significantly improved, being reduced to just 55 g/m² for 24 hours. However, using the CFR1 comparison sample, a notable deterioration (increase) in the ability to absorb water was found in all conditions of Test 1 (60 seconds, 120 seconds, and 300 seconds).

Surprisingly, the INV1 invention sample showed a water vapor transmission (WVTR) value through paper sheets of just 15 g/m² for 24 hours in the standard environmental condition of 23° C. and 50% relative humidity (Test 2), extremely lower than the data obtained from the CFR1 comparison sample (in fact going from 55 to 15 g/m² for 24 hours) and even further reduced compared to the data obtained from the CFR2 comparison sample (going from 115 to 15 g/m² for 24 hours).

The value of 15 g/m² for 24 hours obtained from the INV1 invention sample (which is free of plastic elements) was therefore found to be extremely low, comparable to values obtainable only with bags supplied with plastic elements, which are not recyclable and therefore harmful to the environment.

Even in the tropical conditions of 38° C. and 90% relative humidity, the INV1 invention sample surprisingly showed a water vapor transmission (WVTR) value through the paper sheets that was significantly lower than the data obtained in the same conditions from the CFR1 and CFR2 comparison samples.

At the same time, the INV1 invention sample also showed acceptable values relating to the capacity to absorb water in all conditions of Test 1 (60 seconds, 120 seconds, and 300 seconds).

Therefore, the bag of the present invention, despite being free of intermediate plastic layers, provides excellent moisture absorption results thanks to its structure consisting of a semi-extensible paper layer, onto which a water vapor barrier layer is applied and onto which a hydrophobic paint layer is applied. In fact, the above reported structure of the bag of the invention provided surprising synergistic results, compared to the CFR1 and CFR2 comparison examples where only the water vapor barrier layer (CFR1) or the hydrophobic paint layer (CFR2) were present onto the semi-extensible paper sheet. This results in a long shelf life of the product contained inside the bag.

### Example 4 (Invention)

An invention bag, here referred to as INV2, has the same structure as the outer sheet of the INV1 invention bag seen above. The INV2 invention sample differs from the INV1 invention sample in that the same water vapor barrier layer used in the structure of the outer sheet has also been applied to the semi-extensible paper layer of the internal sheet. The INV2 invention sample showed a water vapor transmission (WVTR) value through the paper sheets equal to only 10 g/m² for 24 hours in the standard environmental condition of 23° C. and 50% relative humidity (Test 2). The value obtained with the same test using the INV1 invention sample is thus further lowered, thanks to the further addition of a water vapor barrier layer on the internal sheet of semi-extensible paper.

Of course, many modifications and variations of the preferred embodiments described above will be apparent to those skilled in the art, while still remaining within the scope of the present invention.

For example, instead of using an outer sheet of semi-extensible paper with a weight of 70 g/m² in the invention bag, it is possible to use weights of 80 and 90 g/m², keeping unchanged the 15 g/m² weight of the water vapor barrier layer applied onto one side of this outer sheet, thus obtaining an overall structure of, respectively, 95 and 105 g/m².

The side of the outer sheet where the water vapor barrier layer is present is smoother than the opposite side of the outer sheet, providing the bag with surface characteristics similar to those of a coated paper. The smoother a surface, the higher quality the paper can be printed on.

Therefore, the present invention is not limited to the preferred embodiments described, illustrated for illustrative and non-limiting purposes only, but is defined by the claims that follow.

1. Bag consisting of an outer paper sheet superimposed on an internal paper sheet, wherein:
each of said outer paper sheet and internal paper sheet has a weight of 70-90 g/m² and is of the semi-extensible type, i.e. it has a tensile strength in the machine direction MD of 6.4-7.8 kN/m and a tensile strength in the transverse direction TD of 4.7-5.8 kN/m measured according to ISO 1924-3:2019,
each of said outer paper sheet and internal paper sheet is coated on one side with a water vapor barrier layer having a weight of 10-20 g/m² measured according to the ISO 536:2019 standard,
said water vapor barrier layer comprises a styrene-based polymer dispersion and at least one pigment composed of at least clay and calcium carbonate,
said water vapor barrier layer is in turn coated with at least one hydrophobic paint layer of the water-based wax type, such as for example a polymeric wax, including at least one element of the isothiazol-3-one type, such as for example 1,2-benzisothiazol-3 (2H)-one, 5-chloro-2-methyl-2H-isothiazol-3-one (CMIT) and/or 2-methyl-2H-isothiazol-3-one, and mixtures thereof.

2. Bag according to claim 1, characterized in that said at least one hydrophobic paint layer has a weight of 4-9 g/m².

3. Bag according to claim 1, wherein each of said outer paper sheet and internal paper sheet of the semi-extensible type has a tensile index in the machine direction MD equal to approximately 75 Nm/g according to the ISO 1924 standard-3:2019.

4. Bag according to claim 1, wherein each of said outer paper sheet and internal paper sheet of the semi-extensible type has an elongation at break in the machine direction MD of about 5.2% and an elongation in the transverse direction TD of 9-9.5% according to ISO 1924-3:2019.

5. Bag according to claim 1, wherein each of said outer paper sheet and internal paper sheet is coated on one side with a water vapor barrier layer having a weight of approximately 15 g/m².

6. Bag according to claim 1, wherein said polymer dispersion comprises styrene-acrylate and/or styrene-butadiene.

7. Bag according to claim 1, wherein said barrier layer is applied to said outer paper sheet and to said internal paper sheet by means of a coating machine in off-line mode.

8. A bag according to claim 7, wherein said coating machine is provided with a blade and an air dryer.

9. Bag according to claim 1, constituted by a main lateral external surface and by at least one lower bottom fixed to said main lateral external surface, wherein each of said main lateral external surface and said lower bottom is constituted by at least two of said internal and outer paper sheets paper superimposed on each other coated with at least one hydrophobic paint layer.

10. Bag according to claim 1, wherein said bag is furthermore constituted by an upper bottom fixed to said main lateral external surface, wherein also said upper bottom is constituted by at least two of said internal and outer paper sheets superimposed one to the other coated with at least one hydrophobic paint layer.
